(19) United States
(12) Patent Application Publication
(10) Pub. No.: US 2024/0130401 A1
(43) Pub. Date: Apr. 25, 2024

(54) DIRECT FED MICROBIALS USING B. COAGULANS

(71) Applicant: MICROBIAL DISCOVERY GROUP, LLC, Franklin, WI (US)

(72) Inventors: Michael R. King, Oak Creek, WI (US); Sona Son, Franklin, WI (US)

(21) Appl. No.: 18/482,000

(22) Filed: Oct. 5, 2023

Related U.S. Application Data
(60) Provisional application No. 63/413,412, filed on Oct. 5, 2022.

(51) Int. Cl. A23K 10/18; A23K 20/195; C12N 1/20

(52) U.S. Cl. CPC A23K 10/18; A23K 20/195; C12N 1/205; C12R 2001/07

(57) ABSTRACT

The invention relates to direct-fed microbials for use in improving the performance of an animal and improving the health of an animal, and combinations thereof. More particularly, the invention relates to isolated Bacillus coagulans strains, and strains having all of the identifying characteristics of these strains, for uses comprising the above-mentioned uses.

### CROSS-REFERENCE TO RELATED APPLICATIONS

This application claims the benefit under 35 USC § 119(e) of U.S. Provisional Application Ser. No. 63/413,412, filed on Oct. 5, 2022, the entire disclosure of which is incorporated herein by reference.

### FIELD OF THE DISCLOSURE

The invention relates to direct-fed microbials for use in improving the performance of an animal and improving the health of an animal, and combinations thereof. More particularly, the invention relates to isolated Bacillus coagulans strains, and strains having all of the identifying characteristics of these strains, for uses comprising the above-mentioned uses.

### BACKGROUND AND SUMMARY OF THE INVENTION

The present invention relates to direct-fed microbial (DFM) compositions and methods for improving the performance of an animal and improving the health of an animal, and combinations thereof. These improvements enhance commercial value of animal populations.

An animal's gastrointestinal tract is constantly challenged by large numbers of bacteria, viruses, and protozoa found in feed, bedding, and the environment. The gastrointestinal tract has a sophisticated system to counter these potential pathogens consisting of physical, chemical, and immunological lines of defense. Beneficial bacteria are an important part of this system. Pathogens, stress, metabolic upset, the use of antimicrobials, and other causes can upset the balance of intestinal bacteria, which may impair digestion and make the animal more susceptible to disease.

Direct-fed microbial products are products that contain live (viable) microorganisms (e.g., bacteria). Over time, many of the direct-fed microbial products previously considered useful for improving animal performance, for example, directly via feed conversion improvements, have lost overall efficacy. Thus, microbial strains are needed that will improve animal performance, including average daily feed intake, average daily gain, feed conversion, nutrient absorption, energy utilization, and overall growth performance.

Applicants have developed a direct-fed microbial composition comprising B. coagulans strains that inhibit inflammation in animals resulting in increased growth performance (e.g., increased nutrient adsorption, increased average daily gain, increased average daily feed intake, and improved feed conversion), improved metabolizable energy (i.e., improved energy utilization), and reduced disease concerns from animal pathogens. The direct-fed microbial compositions described herein offer a commercial benefit by providing all of these properties, or a combination thereof, in a single direct-fed microbial composition. In addition, the direct-fed microbial compositions described herein result in a reduction in the use of antibiotics, and an increase in feed efficiency, which reduces the overall cost of animal feed.

Methods and compositions are provided for inhibiting inflammation in animals resulting in increased growth performance (e.g., increased nutrient adsorption, increased average daily gain, increased average daily feed intake, and improved feed conversion), improved metabolizable energy (i.e., improved energy utilization), and reduced disease concerns from animal pathogens, and combinations thereof. In various embodiments, the animal can be selected from the group consisting of a poultry species, a porcine species, a bovine species, an ovine species, an equine species, and a companion animal. In the embodiment where the animal is a poultry species, the poultry species can be a broiler chicken. In the embodiment where the animal is a porcine species, the porcine species can be selected from the group consisting of a grow finish pig, a nursery pig, a sow, and a breeding stock pig.

In one embodiment, a method of feeding an animal is provided. The method comprises the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof, wherein the Bacillus strain causes an effect selected from the group consisting of improving the performance of the animal and improving the health of the animal, and combinations thereof.

In another embodiment, a method of feeding an animal is provided. The method comprises the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

In various embodiments, the compositions for use in the methods described herein can be a commercial package, a feed additive for an animal feed composition, an additive for the drinking water of an animal, or an animal feed composition (e.g., a complete feed), each comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the EXAMPLES are applicable to any of the following embodiments of the invention described in the numbered clauses below.
1. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof, wherein the Bacillus strain causes an effect selected from the group consisting of improving the performance of the animal and improving the health of the animal, and combinations thereof.
2. The method of clause 1 wherein the animal is selected from the group consisting of a poultry species, a porcine species, a bovine species, an ovine species, an equine species, and a companion animal.
3. The method of clause 2 wherein the poultry species is a broiler chicken.
4. The method of any one of clauses 1 to 3 wherein the effect is improving the performance of the animal.
5. The method of clause 4 wherein the improvement in animal performance is selected from the group consisting of increased nutrient adsorption, increased average daily gain, increased average daily feed intake, decreasing feed conversion, increasing the digestibility of a diet, increasing the metabolizable energy to gross energy ratio, improving consistency of performance, and combinations thereof.
6. The method of clause 2 wherein the porcine species is selected from the group consisting of a grow finish pig, a nursery pig, a sow, and a breeding stock pig.
7. The method of any one of clauses 1 to 6 wherein the effect is improving the health of the animal.
8. The method of any one of clauses 1 to 7 wherein the Bacillus strain produces an enzyme selected from the group consisting of an α-galactosidase, a protease, a lipase, an amylase, a xylanase, a cellulase, and combinations thereof.
9 The method of any one of clauses 1 to 8 wherein at least one of the Bacillus strains has antimicrobial activity.
10. The method of clause 9 wherein the antimicrobial activity is against bacteria selected from the group consisting of E. coli, Salmonella, Staphylococcus, Enterococcus, Clostridia, Campylobacter, and combinations thereof.
11. The method of clause 7 wherein the improvement to the health of the animal is reduced bioburden in the animal.
12. The method of clause 7 wherein the improvement to the health of the animal is increasing the immune response in the animal.
13. The method of any one of clauses 1 to 12 further comprising the step of administering to the animal another bacterial strain selected from the group consisting of another different Bacillus strain, a lactic acid bacterial strain, and combinations thereof.
14. The method of any one of clauses 1 to 13 wherein the strain administered is Bacillus strain BC1 (NRRL No. B-67744), or a strain having all of the identifying characteristics of Bacillus strain BC1 (NRRL No. B-67744), or a combination thereof.
15. The method of any one of clauses 1 to 13 wherein the strain administered is Bacillus strain BCP2 (NRRL No. B-67745), or a strain having all of the identifying characteristics of Bacillus strain BCP2 (NRRL No. B-67745), or a combination thereof.
16. The method of any one of clauses 1 to 13 wherein the strain administered is Bacillus strain BC1 (NRRL No. B-67744).
17. The method of any one of clauses 1 to 13 wherein the strain administered is Bacillus strain BCP2 (NRRL No. B-67745).
18. The method of any one of clauses 1 to 13 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in a single composition.
19. The method of any one of clauses 1 to 13 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in separate compositions.
20. The method of any one of clauses 1 to 19 wherein the Bacillus strain is administered in the feed composition at a dose of about 1.0×10³ CFU/gram of the feed composition to about 5.0×10¹² CFU/gram of the feed composition.
21. The method of any one of clauses 1 to 19 wherein the Bacillus strain is administered in the feed composition at a dose of about 1.0×10³ CFU/gram of the feed composition to about 1.0×10⁷ CFU/gram of the feed composition.
22. The method of any one of clauses 1 to 19 wherein the Bacillus strain is administered in the feed composition at a dose greater than about 7.0×10⁴ CFU/gram of the feed composition.
23. The method of any one of clauses 1 to 22 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.
24. The method of any one of clauses 1 to 23 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.
25. The method of any one of clauses 1 to 24 wherein the animal is a sow and the Bacillus strain is administered during lactation.
26. The method of any one of clauses 1 to 24 wherein the animal is a sow and the Bacillus strain is administered during gestation.
27. The method of any one of clauses 1 to 24 wherein the feed composition is administered daily to the animal.
28. The method of any one of clauses 1 to 27 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
29. A commercial package comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
30. A feed additive for an animal feed comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
31. An additive for the drinking water of an animal comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
32. An animal feed composition comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
33. The commercial package, feed additive, feed composition, or additive for the drinking water of the animal of any one of clauses 29 to 32 wherein the Bacillus strain causes an effect selected from the group consisting of improving the performance of the animal, improving the health of the animal, and combinations thereof.
34. The commercial package, feed additive, feed composition, or additive for the drinking water of the animal of any one of clauses 29 to 33, wherein the Bacillus strain inhibits a pathogen selected from the group consisting of E. coli, Salmonella, Staphylococcus, Enterococcus, Campylobacter, and Clostridium.
35. The feed additive or additive for the drinking water of the animal of clause 30 or 31 in the form of a concentrate.
36. The feed additive or additive for the drinking water of the animal of clause 30 or 31 in the form of a superconcentrate.
37. The feed additive, the feed composition, or the additive for the drinking water of the animal of any one of clauses 30 to 34 in dry form.
38. The feed additive, the feed composition, or the additive for the drinking water of the animal of any one of clauses 30 to 34 in pelleted form.
39. The feed additive, the feed composition, or the additive for the drinking water of the animal of any one of clauses 30 to 34 in powder form.
40. The commercial package, feed additive, feed composition, or additive for the drinking water of the animal of any one of clauses 29 to 34 wherein the strains are in a form selected from the group consisting of a powder, a liquid, a gel, a freeze-dried form, a top-dressing, and a pellet form.
41. The commercial package, feed additive, additive for the drinking water of the animal, or feed composition of any one of clauses 29 to 40 further comprising a carrier for the Bacillus strains.
42. The commercial package, feed additive, additive for the drinking water of the animal, or feed composition of clause 41 wherein the carrier is selected from the group consisting of a bran, rice hulls, a salt, mineral oil, a dextrin, whey, sugar, limestone, dried starch, sodium silico aluminate, vegetable oil, and combinations thereof.
43. The commercial package, feed additive, additive for the drinking water of the animal, or feed composition of any one of clauses 29 to 42 in a bag.
44. The commercial package, feed additive, additive for the drinking water of the animal, or feed composition of clause 43 wherein the bag is a plastic bag.
45. The commercial package, feed additive, additive for the drinking water of the animal, or feed composition of any one of clauses 29 to 44 further comprising instructions for use of one or more of the Bacillus strains.
46. The commercial package, feed additive, or additive for the drinking water of the animal, or the feed composition of any one of clauses 29 to 45 in a 20-pound bag.
47. The commercial package, feed additive, or additive for the drinking water of the animal or the feed composition of any one of clauses 29 to 46 in a 50-pound bag.
48. The commercial package, feed additive, additive for the drinking water of the animal, or feed composition of any one of clauses 29 to 47 in a container for commercial use.
49. The commercial package, feed additive, additive for the drinking water of the animal, or feed composition of clause 48 wherein the container comprises plastic.
50. The commercial package, feed additive, additive for the drinking water of the animal, or feed composition of clause 48 wherein the container comprises paper.
51. The commercial package, feed additive, additive for the drinking water of the animal, or feed composition of any one of clauses 29 to 50 further comprising a binder.
52. The commercial package, feed additive, additive for the drinking water of the animal, or feed composition of clause 51 wherein the binder is selected from the group consisting of clay, yeast cell wall components, aluminum silicate, glucan, and combinations thereof.
53. The commercial package, feed additive, additive for the drinking water of the animal, or feed composition of any one of clauses 29 to 52 in the form of a dietary nutrient composition.
54. The commercial package, feed additive, additive for the drinking water of the animal, or feed composition of any one of clauses 29 to 53 further comprising an exogenously added nutrient component selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.
55. The commercial package, feed additive, additive for the drinking water of the animal, or feed composition of clause 54 wherein the exogenously added component is an enzyme.
56. The commercial package, feed additive, additive for the drinking water of the animal, or feed composition of clause 55 wherein the enzyme is selected from the group consisting of a galactosidase, a protease, a lipase, an amylase, a hemicellulase, an arabinoxylanase, a xylanase, a cellulase, an NSPase, a phytase, and combinations thereof.
57. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
58. The method of clause 57 wherein the animal is selected from the group consisting of a poultry species, a porcine species, a bovine species, an ovine species, an equine species, and a companion animal.
59. The method of clause 58 wherein the poultry species is a broiler chicken.
60. The method of clause 58 wherein the porcine species is selected from the group consisting of a grow finish pig, a nursery pig, a sow, and a breeding stock pig.
61. The method of any one of clauses 57 to 60 wherein the Bacillus strain produces an enzyme selected from the group consisting of an α-galactosidase, a protease, a lipase, an amylase, a xylanase, a cellulase, and combinations thereof.
62 The method of any one of clauses 57 to 61 wherein at least one of the Bacillus strains has antimicrobial activity.
63. The method of clause 62 wherein the antimicrobial activity is against bacteria selected from the group consisting of E. coli, Salmonella, Staphylococcus, Enterococcus, Clostridia, Campylobacter, and combinations thereof.
64. The method of any one of clauses 57 to 63 further comprising the step of administering to the animal another bacterial strain selected from the group consisting of another different Bacillus strain, a lactic acid bacterial strain, and combinations thereof.
65. The method of any one of clauses 57 to 64 wherein the strain administered is Bacillus strain BC1 (NRRL No. B-67744), or a strain having all of the identifying characteristics of Bacillus strain BC1 (NRRL No. B-67744), or a combination thereof.
66. The method of any one of clauses 57 to 64 wherein the strain administered is Bacillus strain BCP2 (NRRL No. B-67745), or a strain having all of the identifying characteristics of Bacillus strain BCP2 (NRRL No. B-67745), or a combination thereof.
67. The method of any one of clauses 57 to 64 wherein the strain administered is Bacillus strain BC1 (NRRL No. B-67744).
68. The method of any one of clauses 57 to 64 wherein the strain administered is Bacillus strain BCP2 (NRRL No. B-67745).
69. The method of any one of clauses 57 to 64 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in a single composition.
70. The method of any one of clauses 57 to 64 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in separate compositions.
71. The method of any one of clauses 57 to 70 wherein the Bacillus strain is administered in the feed composition at a dose of about 1.0×10³ CFU/gram of the feed composition to about 5.0×10¹² CFU/gram of the feed composition.
72. The method of any one of clauses 57 to 70 wherein the Bacillus strain is administered in the feed composition at a dose of about 1.0×10³ CFU/gram of the feed composition to about 1.0×10⁷ CFU/gram of the feed composition.
73. The method of any one of clauses 57 to 72 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.
74. The method of any one of clauses 57 to 73 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.
75. The method of any one of clauses 57 to 74 wherein the animal is a sow and the Bacillus strain is administered during lactation.
76. The method of any one of clauses 57 to 74 wherein the animal is a sow and the Bacillus strain is administered during gestation.
77. The method of any one of clauses 57 to 74 wherein the feed composition is administered daily to the animal.
78. The method of any one of clauses 57 to 74 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.

### BRIEF DESCRIPTION OF THE DRAWINGS

FIGS. 1A-C show relative quantification of expression of inflammatory chemokines and cytokines in the IEC-6 cell line challenged with 10 ng LPS and treated with Bacillus coagulans (BCP2) at 10³, 10⁴, and 10⁵ CFU/ml. Different letters indicate a significant difference (p<0.05).

FIGS. 2A-C show relative quantification of expression of inflammatory cytokines and pathogen receptors in the IEC-6 cell line challenged with 10 ng LPS and treated with Bacillus coagulans (BC1) at 10³, 10⁴, and 10⁵ CFU/ml. Different letters indicate a significant difference (p<0.05).

FIG. 3 shows expression of pathogen receptors and cytokines in pigs exposed to a high bioburden environment and treated with a Bacillus DFM, or a Bacillus DFM and Bacillus coagulans (BC1). Asterisks indicate a value that is significantly different from the negative control (p<0.05). From left to right in each group, the bars are negative control, high bioburden control, high bioburden control+DFM, and high bioburden control+DFM+Bacillus coagulans strain BC1.

FIG. 4 shows body weight, live gain, gain to feed ratio, and feed intake of birds fed Bacillus coagulans.

### DETAILED DESCRIPTION OF ILLUSTRATIVE EMBODIMENTS

Methods and compositions are provided for inhibiting inflammation in animals resulting in increased growth performance (e.g., increased nutrient adsorption, increased average daily gain, increased average daily feed intake, and improved feed conversion), improved metabolizable energy (i.e., improved energy utilization), and reduced disease concerns from animal pathogens, and combinations thereof. In various embodiments, the animal can be selected from the group consisting of a poultry species, a porcine species, a bovine species, an ovine species, an equine species, and a companion animal. In the embodiment where the animal is a poultry species, the poultry species can be a broiler chicken. In the embodiment where the animal is a porcine species, the porcine species can be selected from the group consisting of a grow finish pig, a nursery pig, a sow, and a breeding stock pig.

In one embodiment, a method of feeding an animal is provided. The method comprises the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof, wherein the Bacillus strain causes an effect selected from the group consisting of improving the performance of the animal and improving the health of the animal, and combinations thereof.

In another embodiment, a method of feeding an animal is provided. The method comprises the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

In various embodiments, the compositions for use in the methods described herein can be a commercial package, a feed additive for an animal feed composition, an additive for the drinking water of an animal, or an animal feed composition (e.g., a complete feed), each comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

The following clauses, and combinations thereof, provide various additional illustrative aspects of the invention described herein. The various embodiments described in this section titled “DETAILED DESCRIPTION OF ILLUSTRATIVE EMBODIMENTS” are applicable to any of the following embodiments of the invention described in the numbered clauses below.
1. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof, wherein the Bacillus strain causes an effect selected from the group consisting of improving the performance of the animal and improving the health of the animal, and combinations thereof.
2. The method of clause 1 wherein the animal is selected from the group consisting of a poultry species, a porcine species, a bovine species, an ovine species, an equine species, and a companion animal.
3. The method of clause 2 wherein the poultry species is a broiler chicken.
4. The method of any one of clauses 1 to 3 wherein the effect is improving the performance of the animal.
5. The method of clause 4 wherein the improvement in animal performance is selected from the group consisting of increased nutrient adsorption, increased average daily gain, increased average daily feed intake, decreasing feed conversion, increasing the digestibility of a diet, increasing the metabolizable energy to gross energy ratio, improving consistency of performance, and combinations thereof.
6. The method of clause 2 wherein the porcine species is selected from the group consisting of a grow finish pig, a nursery pig, a sow, and a breeding stock pig.
7. The method of any one of clauses 1 to 6 wherein the effect is improving the health of the animal.
8. The method of any one of clauses 1 to 7 wherein the Bacillus strain produces an enzyme selected from the group consisting of an α-galactosidase, a protease, a lipase, an amylase, a xylanase, a cellulase, and combinations thereof.
9 The method of any one of clauses 1 to 8 wherein at least one of the Bacillus strains has antimicrobial activity.
10. The method of clause 9 wherein the antimicrobial activity is against bacteria selected from the group consisting of E. coli, Salmonella, Staphylococcus, Enterococcus, Clostridia, Campylobacter, and combinations thereof.
11. The method of clause 7 wherein the improvement to the health of the animal is reduced bioburden in the animal.
12. The method of clause 7 wherein the improvement to the health of the animal is increasing the immune response in the animal.
13. The method of any one of clauses 1 to 12 further comprising the step of administering to the animal another bacterial strain selected from the group consisting of another different Bacillus strain, a lactic acid bacterial strain, and combinations thereof.
14. The method of any one of clauses 1 to 13 wherein the strain administered is Bacillus strain BC1 (NRRL No. B-67744), or a strain having all of the identifying characteristics of Bacillus strain BC1 (NRRL No. B-67744), or a combination thereof.
15. The method of any one of clauses 1 to 13 wherein the strain administered is Bacillus strain BCP2 (NRRL No. B-67745), or a strain having all of the identifying characteristics of Bacillus strain BCP2 (NRRL No. B-67745), or a combination thereof.
16. The method of any one of clauses 1 to 13 wherein the strain administered is Bacillus strain BC1 (NRRL No. B-67744).
17. The method of any one of clauses 1 to 13 wherein the strain administered is Bacillus strain BCP2 (NRRL No. B-67745).
18. The method of any one of clauses 1 to 13 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in a single composition.
19. The method of any one of clauses 1 to 13 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in separate compositions.
20. The method of any one of clauses 1 to 19 wherein the Bacillus strain is administered in the feed composition at a dose of about 1.0×10³ CFU/gram of the feed composition to about 5.0×10¹² CFU/gram of the feed composition.
21. The method of any one of clauses 1 to 19 wherein the Bacillus strain is administered in the feed composition at a dose of about 1.0×10³ CFU/gram of the feed composition to about 1.0×10⁷ CFU/gram of the feed composition.
22. The method of any one of clauses 1 to 19 wherein the Bacillus strain is administered in the feed composition at a dose greater than about 7.0×10⁴ CFU/gram of the feed composition.
23. The method of any one of clauses 1 to 22 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.
24. The method of any one of clauses 1 to 23 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.
25. The method of any one of clauses 1 to 24 wherein the animal is a sow and the Bacillus strain is administered during lactation.
26. The method of any one of clauses 1 to 24 wherein the animal is a sow and the Bacillus strain is administered during gestation.
27. The method of any one of clauses 1 to 24 wherein the feed composition is administered daily to the animal.
28. The method of any one of clauses 1 to 27 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
29. A commercial package comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
30. A feed additive for an animal feed comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
31. An additive for the drinking water of an animal comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
32. An animal feed composition comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
33. The commercial package, feed additive, feed composition, or additive for the drinking water of the animal of any one of clauses 29 to 32 wherein the Bacillus strain causes an effect selected from the group consisting of improving the performance of the animal, improving the health of the animal, and combinations thereof.
34. The commercial package, feed additive, feed composition, or additive for the drinking water of the animal of any one of clauses 29 to 33, wherein the Bacillus strain inhibits a pathogen selected from the group consisting of E. coli, Salmonella, Staphylococcus, Enterococcus, Campylobacter, and Clostridium.
35. The feed additive or additive for the drinking water of the animal of clause 30 or 31 in the form of a concentrate.
36. The feed additive or additive for the drinking water of the animal of clause 30 or 31 in the form of a superconcentrate.
37. The feed additive, the feed composition, or the additive for the drinking water of the animal of any one of clauses 30 to 34 in dry form.
38. The feed additive, the feed composition, or the additive for the drinking water of the animal of any one of clauses 30 to 34 in pelleted form.
39. The feed additive, the feed composition, or the additive for the drinking water of the animal of any one of clauses 30 to 34 in powder form.
40. The commercial package, feed additive, feed composition, or additive for the drinking water of the animal of any one of clauses 29 to 34 wherein the strains are in a form selected from the group consisting of a powder, a liquid, a gel, a freeze-dried form, a top-dressing, and a pellet form.
41. The commercial package, feed additive, additive for the drinking water of the animal, or feed composition of any one of clauses 29 to 40 further comprising a carrier for the Bacillus strains.
42. The commercial package, feed additive, additive for the drinking water of the animal, or feed composition of clause 41 wherein the carrier is selected from the group consisting of a bran, rice hulls, a salt, mineral oil, a dextrin, whey, sugar, limestone, dried starch, sodium silico aluminate, vegetable oil, and combinations thereof.
43. The commercial package, feed additive, additive for the drinking water of the animal, or feed composition of any one of clauses 29 to 42 in a bag.
44. The commercial package, feed additive, additive for the drinking water of the animal, or feed composition of clause 43 wherein the bag is a plastic bag.
45. The commercial package, feed additive, additive for the drinking water of the animal, or feed composition of any one of clauses 29 to 44 further comprising instructions for use of one or more of the Bacillus strains.
46. The commercial package, feed additive, or additive for the drinking water of the animal, or the feed composition of any one of clauses 29 to 45 in a 20-pound bag.
47. The commercial package, feed additive, or additive for the drinking water of the animal or the feed composition of any one of clauses 29 to 46 in a 50-pound bag.
48. The commercial package, feed additive, additive for the drinking water of the animal, or feed composition of any one of clauses 29 to 47 in a container for commercial use.
49. The commercial package, feed additive, additive for the drinking water of the animal, or feed composition of clause 48 wherein the container comprises plastic.
50. The commercial package, feed additive, additive for the drinking water of the animal, or feed composition of clause 48 wherein the container comprises paper.
51. The commercial package, feed additive, additive for the drinking water of the animal, or feed composition of any one of clauses 29 to 50 further comprising a binder.
52. The commercial package, feed additive, additive for the drinking water of the animal, or feed composition of clause 51 wherein the binder is selected from the group consisting of clay, yeast cell wall components, aluminum silicate, glucan, and combinations thereof.
53. The commercial package, feed additive, additive for the drinking water of the animal, or feed composition of any one of clauses 29 to 52 in the form of a dietary nutrient composition.
54. The commercial package, feed additive, additive for the drinking water of the animal, or feed composition of any one of clauses 29 to 53 further comprising an exogenously added nutrient component selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.
55. The commercial package, feed additive, additive for the drinking water of the animal, or feed composition of clause 54 wherein the exogenously added component is an enzyme.
56. The commercial package, feed additive, additive for the drinking water of the animal, or feed composition of clause 55 wherein the enzyme is selected from the group consisting of a galactosidase, a protease, a lipase, an amylase, a hemicellulase, an arabinoxylanase, a xylanase, a cellulase, an NSPase, a phytase, and combinations thereof.
57. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.
58. The method of clause 57 wherein the animal is selected from the group consisting of a poultry species, a porcine species, a bovine species, an ovine species, an equine species, and a companion animal.
59. The method of clause 58 wherein the poultry species is a broiler chicken.
60. The method of clause 58 wherein the porcine species is selected from the group consisting of a grow finish pig, a nursery pig, a sow, and a breeding stock pig.
61. The method of any one of clauses 57 to 60 wherein the Bacillus strain produces an enzyme selected from the group consisting of an α-galactosidase, a protease, a lipase, an amylase, a xylanase, a cellulase, and combinations thereof.
62 The method of any one of clauses 57 to 61 wherein at least one of the Bacillus strains has antimicrobial activity.
63. The method of clause 62 wherein the antimicrobial activity is against bacteria selected from the group consisting of E. coli, Salmonella, Staphylococcus, Enterococcus, Clostridia, Campylobacter, and combinations thereof.
64. The method of any one of clauses 57 to 63 further comprising the step of administering to the animal another bacterial strain selected from the group consisting of another different Bacillus strain, a lactic acid bacterial strain, and combinations thereof.
65. The method of any one of clauses 57 to 64 wherein the strain administered is Bacillus strain BC1 (NRRL No. B-67744), or a strain having all of the identifying characteristics of Bacillus strain BC1 (NRRL No. B-67744), or a combination thereof.
66. The method of any one of clauses 57 to 64 wherein the strain administered is Bacillus strain BCP2 (NRRL No. B-67745), or a strain having all of the identifying characteristics of Bacillus strain BCP2 (NRRL No. B-67745), or a combination thereof.
67. The method of any one of clauses 57 to 64 wherein the strain administered is Bacillus strain BC1 (NRRL No. B-67744).
68. The method of any one of clauses 57 to 64 wherein the strain administered is Bacillus strain BCP2 (NRRL No. B-67745).
69. The method of any one of clauses 57 to 64 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in a single composition.
70. The method of any one of clauses 57 to 64 wherein at least two of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), or combinations thereof, are administered in combination in separate compositions.
71. The method of any one of clauses 57 to 70 wherein the Bacillus strain is administered in the feed composition at a dose of about 1.0×10³ CFU/gram of the feed composition to about 5.0×10¹² CFU/gram of the feed composition.
72. The method of any one of clauses 57 to 70 wherein the Bacillus strain is administered in the feed composition at a dose of about 1.0×10³ CFU/gram of the feed composition to about 1.0×10⁷ CFU/gram of the feed composition.
73. The method of any one of clauses 57 to 72 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.
74. The method of any one of clauses 57 to 73 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, and combinations thereof.
75. The method of any one of clauses 57 to 74 wherein the animal is a sow and the Bacillus strain is administered during lactation.
76. The method of any one of clauses 57 to 74 wherein the animal is a sow and the Bacillus strain is administered during gestation.
77. The method of any one of clauses 57 to 74 wherein the feed composition is administered daily to the animal.
78. The method of any one of clauses 57 to 74 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.

In various embodiments, the animal to which a feed additive, a feed composition, or drinking water as described herein is administered can be selected from the group consisting of a poultry species, a porcine species, a bovine species, an ovine species, an equine species, a companion animal, or a human. In the embodiment where the animal is a companion animal, the companion animal can be, for example, a canine species or a feline species. In the embodiment where the animal is a porcine species, the porcine species can be selected from the group consisting of a grow finish pig, a nursery pig, a sow, and a breeding stock pig. In various exemplary embodiments, the animal can be selected from the group consisting of a chicken (e.g., a broiler or a layer), a pig, a horse, a pony, a cow, a turkey, a goat, a sheep, a quail, a pheasant, an ostrich, a duck, a fish (e.g., a tilapia, a catfish, a flounder, or a salmon), a crustacean (e.g., a shrimp or a crab), and combinations thereof. In various embodiments described herein, the feed additive, the feed composition, or the additive for the drinking water of an animal can be a dietary nutrient composition (e.g., a probiotic composition). In one embodiment, the commercial package described herein can contain a dietary nutrient composition comprising the Bacillus coagulans strains described herein.

In one embodiment of the invention, an effective amount of the Bacillus strain can be administered to improve the performance of the animal or improve the health of the animal, or combinations thereof. By “effective amount” is meant an amount of the Bacillus strain (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof) capable of improving the performance of the animal or improving the health of the animal, or combinations thereof, by any mechanism, including those described herein.

In embodiments described herein wherein the compositions of the present invention comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, are administered to an animal, the compositions are preferably administered to animals orally in a feed composition or in drinking water, but any other effective method of administration known to those skilled in the art may be utilized. In one illustrative embodiment, the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, are provided in the form of an additive for addition to a feed composition (i.e., food) or to the drinking water of an animal.

In another illustrative embodiment, the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, are provided in the form of a feed additive for addition to a feed composition. The feed composition may contain Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, in a mixture with an animal feed blend, including any art-recognized animal feed blend or any animal feed blend described herein. As used herein, “feed composition” or “animal feed composition” means a feed composition comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, in a mixture with an animal feed blend, and, optionally any other components that could be used in a feed composition, including other different bacterial strains, such as other Bacillus strains or Lactobacillus strains.

Any animal feed blend, including those known in the art and those described herein, may be used in accordance with the methods and compositions described in this patent application, such as rapeseed meal, cottonseed meal, soybean meal, cornmeal, barley, wheat, silage, and haylage. In various embodiments, the animal feed blend can be supplemented with Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, but other ingredients may optionally be added to the animal feed blend, including other different bacterial strains, such as other Bacillus strains or Lactobacillus strains.

In various illustrative embodiments, optional ingredients of the animal feed blend include sugars and complex carbohydrates such as both water-soluble and water-insoluble monosaccharides, disaccharides, and polysaccharides. Other optional ingredients include dried distillers grain solubles, fat (e.g., crude fat), phosphorous, sodium bicarbonate, limestone, salt, phytate, calcium, sodium, sulfur, magnesium, potassium, copper, iron, manganese, zinc, ash, fish oil, an oil derived from fish meal, raw seed (e.g., flaxseed), an antioxidant, and starch. In another embodiment, minerals may be added in the form of a mineral premix.

Optional amino acid ingredients that may be added to the animal feed blend are arginine, histidine, isoleucine, leucine, lysine, cysteine, methionine, phenylalanine, threonine, tryptophan, valine, tyrosine ethyl HCl, alanine, aspartic acid, sodium glutamate, glycine, proline, serine, cysteine ethyl HCl, and analogs, and salts thereof. Vitamins that may be optionally added are thiamine HCl, riboflavin, pyridoxine HCl, niacin, niacinamide, inositol, choline chloride, calcium pantothenate, biotin, folic acid, ascorbic acid, and vitamins A, B, K, D, E, and the like. In another embodiment, vitamins may be added in the form of a vitamin premix. In yet another embodiment, protein ingredients may be added to the animal feed blend and include protein obtained from meat meal, bone meal, or fish meal, liquid or powdered egg, fish solubles, crude protein, and the like.

In another illustrative aspect, any medicament ingredients known in the art may be added to the animal feed blend or to an additive for the drinking water of the animal, such as antibiotics. In various embodiments, the antibiotic is selected from the group consisting of ampicillin, chloramphenicol, ciprofloxacin, clindamycin, tetracycline, chlortetracycline, Denagard™ (i.e., tiamulin), BMD™ (i.e., bacitracin methylene disalicylate), Carbadox™ (i.e., carbadox), Stafac™ (i.e., virginiamycin), erythromycin, levofloxacin, trimethoprim/sulfamethoxazole, trimethoprim, daptomycin, rifampicin, Tylan™ (i.e., tylosin), Pulmotil™ (i.e., tilmicosin), vancomycin, avilamycin (Kavault™), gentamycin, neomycin, and combinations thereof. In another embodiment, the animal feed blend, the feed composition, the feed additive, or the additive for the drinking water of the animal may contain no antibiotics.

In another illustrative embodiment, one or more enzymes may be added to the animal feed blend. In various embodiments, the enzymes that may be added include a galactosidase, a phytase, a protease, a lipase, an amylase, a hemicellulase, an arabinoxylanase, a xylanase, a cellulase, an NSPase, combinations thereof, and any other enzyme that improves the effectiveness of the feed composition for improving the performance or health of the animal. In yet another embodiment, yeast, fungi (e.g., Aspergillus or Trichoderma), or micronutrients may be added to the animal feed. Any of the ingredients described above that are suitable for addition to an additive for the drinking water of the animal may be added as a component of the additive for the drinking water of the animal as described herein.

In various illustrative embodiments, the Bacillus strain (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof), or any other bacterial strains added in addition to Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can be administered in the feed composition at a dose of about 1.0×10³ CFU/gram of the feed composition to about 5.0×10¹² CFU/gram of the feed composition or at a dose of about 1.0×10³ CFU/gram of the feed composition to about 1.0×10⁷ CFU/gram of the feed composition. In other embodiments, the Bacillus strain (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof) is administered in the feed composition at a dose greater than about 1.0×10³ CFU/gram of the feed composition, at a dose greater than about 1.1×10³ CFU/gram of the feed composition, at a dose greater than about 1.25×10³ CFU/gram of the feed composition, at a dose greater than about 1.5×10³ CFU/gram of the feed composition, at a dose greater than about 1.75×10³ CFU/gram of the feed composition, at a dose greater than about 1.0×10⁴ CFU/gram of the feed composition, at a dose greater than about 2.0×10⁴ CFU/gram of the feed composition, at a dose greater than about 3.0×10⁴ CFU/gram of the feed composition, at a dose greater than about 4.0×10⁴ CFU/gram of the feed composition, at a dose greater than about 5.0×10⁴ CFU/gram of the feed composition, at a dose greater than about 6.0×10⁴ CFU/gram of the feed composition, at a dose greater than about 7.0×10⁴ CFU/gram of the feed composition, at a dose greater than about 8.0×10⁴ CFU/gram of the feed composition, at a dose greater than about 1.0×10⁵ CFU/gram of the feed composition, at a dose greater than about 1.0×10⁶ CFU/gram of the feed composition, at a dose greater than about 1.0×10⁷ CFU/gram of the feed composition, at a dose greater than about 1.0×10⁸ CFU/gram of the feed composition, at a dose greater than about 1.0×10⁹ CFU/gram of the feed composition, at a dose greater than about 1.0×10¹⁰ CFU/gram of the feed composition, at a dose greater than about 1.0×10¹¹ CFU/gram of the feed composition, or at a dose greater than about 1.0×10¹² CFU/gram of the feed composition. In yet another embodiment, the Bacillus strain (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof) is administered in the feed composition at a dose of about 7×10⁴ CFU/gram of the feed composition.

In various embodiments, the Bacillus coagulans strain (e.g., Bacillus strain BC1 and/or BCP2) for use in accordance with the methods and compositions described herein can be selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof. These strains are Bacillus coagulans strains. Bacillus strain MDGBC1 (strain BC1) and Bacillus strain MDGBCP2 (strain BCP2) were deposited on Feb. 6, 2019 at the Agricultural Research Service Culture Collection (NRRL), National Center for Agricultural Utilization Research, Agricultural Research Service, USDA, 1815 North University Street, Peoria, Illinois 61604-3999, and were given accession numbers B-67744 and B-67745, respectively. The deposits were made under the provisions of the Budapest Treaty on the International Recognition of the Deposit of Microorganisms for the Purposes of Patent Procedure. The NRRL strain designations are MDGBC1 and MDGBCP2, which are equivalent to Bacillus coagulans strain BC1 and BCP2, respectively, as referred to in this application.

Any of these strains can be administered alone or in combination in the form of a feed composition (e.g., a complete feed comprising an animal feed blend) or drinking water for an animal. In one embodiment, multiple strains are administered in combination in a single composition. In another embodiment, multiple strains are administered in combination in separate compositions.

In another embodiment, one or more of the Bacillus strains described in the preceding paragraphs (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof) can be administered to the animal along with another different bacterial strain selected from the group consisting of another Bacillus strain, a lactic acid bacterial strain, and combinations thereof. In yet another embodiment, one or more of the Bacillus strains described in the preceding paragraphs (e.g., Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof) can be administered to the animal along with any other different bacterial strain effective to improve the performance or health of the animal.

As used herein “a strain having all of the identifying characteristics of” Bacillus strain BC1 (NRRL No. B-67744) or Bacillus strain BCP2 (NRRL No. B-67745) can be a mutant strain having all of the identifying characteristics of Bacillus strain BC1 or Bacillus strain BCP2 (e.g., a DNA fingerprint based on DNA analysis that corresponds to the DNA fingerprint of Bacillus strain BC1 or Bacillus strain BCP2, enzyme activities that correspond to Bacillus strain BC1 or Bacillus strain BCP2, antimicrobial activity that corresponds to Bacillus strain BC1 or Bacillus strain BCP2, antibiotic sensitivity and tolerance profiles that correspond to Bacillus strain BC1 or Bacillus strain BCP2, or combinations thereof). In alternate embodiments, the mutation can be a natural mutation, or a genetically engineered mutation. In another embodiment, “a strain having all of the identifying characteristics of” Bacillus strain BC1 or Bacillus strain BCP2 can be a strain, for example, produced by isolating one or more plasmids from Bacillus strain BC1 or Bacillus strain BCP2 and introducing the one or more plasmids into another bacterium, such as another Bacillus strain, as long as the one or more plasmids contain DNA that provides the identifying characteristics of Bacillus strain BC1 or Bacillus strain BCP2 (e.g., a DNA fingerprint based on DNA analysis that corresponds to the DNA fingerprint of Bacillus strain BC1 or Bacillus strain BCP2).

The feed composition or drinking water comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, may be administered to the animal for any time period that is effective to improve the performance of the animal or improve the health of the animal, or combinations thereof. For example, in one embodiment the feed composition or drinking water may be provided to the animal daily. In an alternate embodiment, the feed composition or drinking water may be administered to the animal during lactation and/or during gestation. The time periods for administration of the feed composition or drinking water described above are non-limiting examples and it should be appreciated that any time period or administration schedule determined to be effective to improve the performance of the animal or improve the health of the animal, or combinations thereof, may be used.

As described herein, one of the method embodiments is a method of feeding an animal by administering to the animal a feed composition or drinking water comprising an effective amount of an additive comprising an isolated Bacillus strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, wherein the Bacillus strain causes an effect selected from the group consisting of improving the performance of the animal and/or improving the health of the animal, and combinations thereof.

In the embodiment where the effect is improving the performance of the animal, the improvement in animal performance can be selected from the group consisting of decreasing feed conversion (e.g., reducing the feed to gain ratio (F/G)), increasing average daily feed intake (ADFI), increasing average daily gain (ADG), improving consistency of performance, improving or increasing digestibility of a diet, improving or increasing the metabolizable energy to gross energy ratio, increasing nutrient adsorption, and combinations thereof. The improvements can be relative to an animal not fed the bacterial strain. In one embodiment, Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can increase the digestibility of a diet by producing enzymes that increase the digestibility of consumed nutrients where the enzymes are selected from the group consisting of an α-galactosidase, a protease, a phytase, a lipase, an amylase, a xylanase, a cellulase, and combinations thereof. The enzyme can also be any other enzyme that degrades long chain fatty acids, such as enzymes that degrade stearic, palmitic, and/or oleic acid, but not limited to these fatty acids. Such an increase in digestibility of a diet leads to improvements in animal performance selected from the group consisting of decreasing feed conversion (e.g., reducing the feed to gain ratio (F/G)), increasing average daily feed intake (ADFI), increasing average daily gain (ADG), improving consistency of performance of the animal (e.g., reducing variation in performance such as reducing variation and increasing uniformity in F/G, ADFI, and ADG), improving the metabolizable energy to gross energy ratio, and combinations thereof.

In the embodiment where the effect is improving the health of the animal, the improvement can result from a mechanism including, but not limited to, antimicrobial activity of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof. In various embodiments, the antimicrobial activity is against bacteria selected from the group consisting of E. coli, Salmonella, Staphylococcus, Enterococcus, Clostridia, Campylobacter, and combinations thereof. Thus, Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof can improve gut health of the animal, and reduce pathogens in the animal, and the animal's environment. In yet another embodiment, Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can reduce bioburden in an animal or increase the immune response in an animal to improve the health of the animal. The improvements described herein can be relative to an animal not fed the bacterial strain.

In this method embodiment, the method can improve the health of the animal by improving the animal's environment by effects selected from the group consisting of reducing respiratory problems of the animal, improving gut health of the animal, improving consistency of performance of the animal, reducing diseases related to environmental toxicity in the animal, and reducing pathogens in the animal. In an embodiment where the animal is a poultry species, the method can improve the health of the animal by an effect selected from the group consisting of reducing respiratory problems of the poultry species, reducing breast blisters of the poultry species, improving consistency of performance of the poultry species, and reducing damage to the feet of the poultry species. These mechanisms of improvement to the health of the animal are non-limiting examples.

In additional embodiments of the invention, compositions comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof are provided. In one embodiment, a commercial package is provided comprising an isolated Bacillus strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), and combinations thereof.

In another embodiment, a feed additive for an animal feed is provided comprising an isolated Bacillus strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), and combinations thereof.

In yet another embodiment, an additive for the drinking water of an animal is provided comprising an isolated Bacillus strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), and combinations thereof.

In yet another illustrative aspect of the invention, an animal feed composition is provided comprising an isolated Bacillus strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), and combinations thereof.

In one embodiment, the feed additive for addition to an animal feed blend to produce a complete feed composition can be mixed with the animal feed blend, for example, with an automated micro-nutrient delivery system, or, for example, by hand-weighing and addition to achieve any of the doses of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, described herein, for administration to the animal in the form of a complete feed composition. The mixing can also be done by any other suitable method known in the art for combining direct-fed microbials with an animal feed blend to obtain a uniform mixture. In various embodiments, the mixing can be done for any suitable time period (e.g., about 1 to about 4 minutes).

In the embodiment where Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, are in the form of an additive for the drinking water of the animal, the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can be in the form of, for example, a powder, a liquid, a gel, a freeze-dried form, a top-dressing, or pellets, and can be mixed with the drinking water using any suitable method known in the art to achieve any of the doses of Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, described herein, for administration to the animal in the drinking water of the animal.

Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can also be fed directly to the animal orally (i.e., by oral insertion) in the form of a powder, a liquid, a gel, a freeze-dried form, a top-dressing, or a pellet.

In any of the composition embodiments described herein, the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can cause an effect selected from the group consisting of improving the performance of the animal or improving the health of the animal, and combinations thereof. The commercial package, feed additive, feed composition, or additive for the drinking water of the animal described herein can also inhibit a pathogen selected from the group consisting of E. coli, Salmonella, Staphylococcus, Enterococcus, Clostridia, Campylobacter, and combinations thereof. These effects are non-limiting examples of the types of effects Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can cause.

In one illustrative aspect, the feed additive, the additive for the drinking water of the animal, or the feed composition can be in the form of a commercial package, such as a dietary nutrient composition (e.g., a probiotic composition). In another illustrative embodiment, the feed additive or additive for the drinking water of the animal, or the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, in the commercial package can be in the form of a concentrate (e.g., about 1×10⁸ to about 5×10⁹ CFU/g) or a superconcentrate (e.g., about 1×10¹⁰ to about 5×10¹² CFU/g). In another embodiment, the feed additive, feed composition, or additive for the drinking water of the animal, or the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, in a composition in a commercial package, can be in a dry form (e.g., a powder), a pelleted form, a liquid form, a freeze-dried form, in the form of a top-dressing, or in the form of a gel, or any other suitable form.

In another illustrative embodiment, the commercial package, feed additive, additive for the drinking water of the animal, or feed composition can further comprise a carrier for the Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof. The carrier can be selected from the group consisting of a bran, rice hulls, a salt, mineral oil, a dextrin (e.g., maltodextrin), whey, sugar, limestone, dried starch, sodium silico aluminate, vegetable oil, and combinations thereof. In another embodiment, the carrier can be any suitable carrier known in the art for a direct-fed microbial. The carrier is exogenously added to the bacterial strain (i.e., not naturally present or not present in nature with the bacterial strain). In another embodiment, the commercial package, feed additive, additive for the drinking water of the animal, or feed composition can further comprise a binder such as clay, yeast cell wall components, aluminum silicate, glucan, or other known binders. The binder is exogenously added to the bacterial strain (i.e., not naturally present or not present in nature with the bacterial strain).

In yet other embodiments, the commercial package, feed additive, additive for the drinking water of the animal, or feed composition comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, is in a container for commercial use. In various embodiments the container can be, for example, a bag (e.g., a 20-pound bag, a 50-pound bag, a 2-ounce bag, a 1-pound bag, or a 1-kilogram bag), a pouch, a drum, a bottle, or a box. In illustrative aspects, the container for the commercial package, feed additive, additive for the drinking water of the animal, or feed composition comprising Bacillus strain BC1 (NRRL No. B-67744), Bacillus strain BCP2 (NRRL No. B-67745), a strain having all of the identifying characteristics of Bacillus strain BC1 (NRRL No. B-67744), and/or a strain having all of the identifying characteristics of Bacillus strain BCP2 (NRRL No. B-67745), or combinations thereof, can comprise plastic, metal, foil, paper, fiber, or cardboard (e.g., a plastic pail, a paper bag, a foil bag, a fiber drum, etc.). The commercial package, feed additive, additive for the drinking water of the animal, or feed composition can further comprise instructions for use of one or more of the Bacillus strains.

In one aspect, the commercial package, feed additive, additive for the drinking water of the animal, or feed composition described herein can further comprise an exogenously added nutrient component (i.e., a nutrient component not present with the bacterial strain in nature) selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.

In one embodiment, the exogenously added nutrient component is an enzyme and the enzyme is selected from the group consisting of a galactosidase, a protease, a lipase, an amylase, a hemicellulase, an arabinoxylanase, a xylanase, a cellulase, an NSPase, a phytase, and combinations thereof.

The following examples are for illustrative purposes only. The examples are non-limiting, and are not intended to limit the invention in any way.

### Example 1

### Immune Modulation by Bacillus coagulans In Vitro

The IEC-6 rat intestinal epithelial cell line was challenged with 10 ng of LPS and treated with 10³, 10⁴, or 10⁵ CFU/ml of Bacillus coagulans strain BCP2 or Bacillus coagulans strain BC1. The cell line was incubated at 37° C. in 5% CO₂ for 2 hours. RNA was extracted from the IEC-6 cells, and qPCR was used to measure expression of immune markers, including inflammatory cytokines, chemokines, and pathogen recognition receptors.

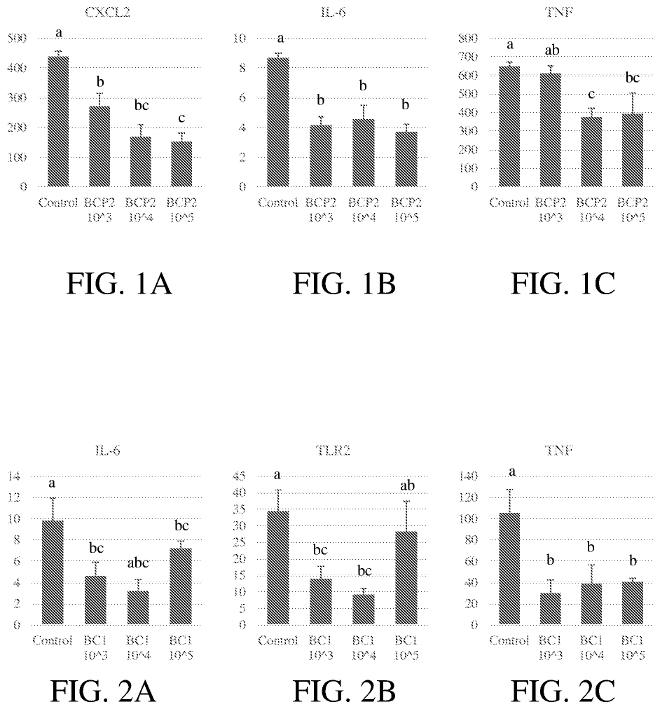

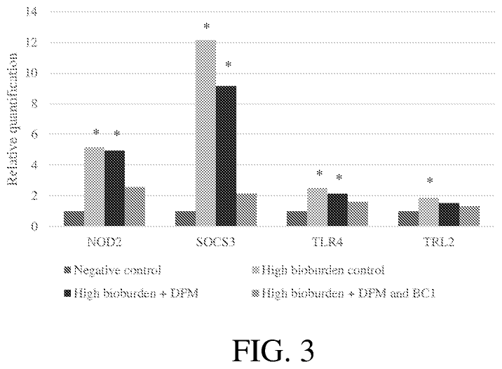

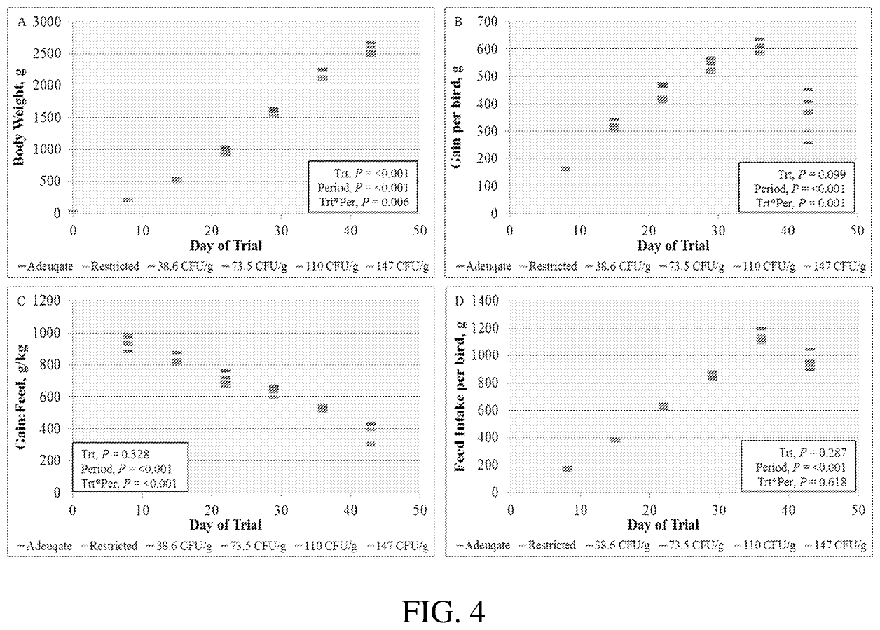

FIGS. 1A-C show Bacillus coagulans strain BCP2 significantly reduced expression of inflammatory immune markers CXCL2, IL-6, and TNF compared to the LPS challenged control. FIGS. 2A-C show Bacillus coagulans strain BC1 significantly reduced expression of inflammatory markers IL-6, TLR2, and TNF compared to the LPS challenged control. The anti-inflammatory effect of both strains was similar at a range of doses from 10³ to 10⁵ CFU/ml.

### Example 2

### In Vivo Animal Trial

Pigs were exposed to management practices and diet consistent with a commercial setting, resulting in an environment with a higher microbial bioburden than the negative control group. The high bioburden animals were fed either a Bacillus DFM without the Bacillus coagulans strain BC1 described herein, or a Bacillus DFM with the Bacillus coagulans strain BC1 described herein for 24 weeks. RNA was extracted from whole blood samples and qPCR was used to measure expression of immune markers.

FIG. 3 shows the high bioburden control group had significantly higher expression of immune markers NOD2, SOCS3, TLR4, and TLR2 than the negative control group, indicating the microbial bioburden induced an immune response. The high bioburden group fed the Bacillus DFM also had significantly higher expression of markers NOD2, SOCS3, and TLR4 than the negative control. The high bioburden group fed the Bacillus DFM with Bacillus coagulans strain BC1 did not differ from the negative control, suggesting Bacillus coagulans BC1 mitigated inflammation caused by the high microbial bioburden.

### Example 3

### Evaluation of Bacillus coagulans in Broilers to Six Weeks Post-Hatch

Broilers were fed both nutrient adequate and nutrient restricted diets up to 6 weeks post-hatch to determine the efficacy of Bacillus coagulans to improve growth performance and feed efficiency. The experimental treatment groups included Treatment A: Positive control nutrient adequate & high fat diet devoid of DFM; Treatment B: Negative control nutrient restricted diet devoid of DFM (CP, AA, & ME reduced 5%); Treatment C: As B+3.68×10¹ CFU/g Bacillus coagulans (0.5×Trt D); Treatment D: As B+7.35×10¹ CFU/g Bacillus coagulans (repeat/validation of 16-P092 as 1×); Treatment E: As B+1.10×10² CFU/g Bacillus coagulans (1.5×Trt D); and Treatment F: As B+1.47×10² CFU/g Bacillus coagulans (2.0×Trt D).

Birds received cocci and Vaccine 2 prior to starting on experimental trial. Birds were allotted to the experiment following SOP allotment procedures using six (6) similar weight pens. At the start of the study, pens were randomly allotted to dietary treatment from within replicate. Pens remained on treatment until experiment end. Daily management followed standard operating procedures when feeding, watering, and monitoring for health decline in the birds. Unit temperature was set at 95° F. and lowered 1° daily starting from day 0 until the temperature reached 85° F., where it was maintained. Water troughs were inspected daily, and cleaned and refreshed if needed. Measurements included total pen weights (at allotment—day 0—and weekly thereafter); feed disappearance (at allotment—day 0—and weekly thereafter); live gain, feed intake, and feed efficiency; morbidity and mortality; and high and low temperatures (along with HOBO device).

The Treatment A diet was formulated to be adequate in EAA, aP, Ca, Na, Mn, and Cu using the Ross Nutrient Specification manual (2014). Treatment B-F diets included a basal restricted diet and ingredient premix. Diets were budgeted by day (Table 2a). Final diets were manufactured at DEO with corn, SBM, DDGS, protein sources, VTMs, and crystalline AA. Final experimental diets were sampled from the manufactured feed in each feeder. Samples of each diet were stored for proximate and AA analysis.

Initial data analysis was performed for all metrics to determine normality of distribution and outliers (±>3 standard deviations from the grand mean). Statistical (SAS) procedures utilized “Pen” as the experimental unit, “Treatment” as the fixed effect, and “Replicate” as the random effect. Body weights, growth rates, feed intakes, and feed efficiency were analyzed using the Mixed procedure of SAS. Means separation used pdiff as single degree-of-freedom comparisons. Serial (repeated) body weights, growth rates, feed intakes and feed efficiency were analyzed using the Mixed procedure of SAS. Repeated Effects (Period)—Covariance matrix modeling (TOEP, CS, AR(1), UN) was run for each matrix; Matrix effecting AICC value closest to zero (0) was used for final analysis. Means separation (Orthogonal single degree-of-freedom comparisons)—Main effects of treatment: Same as for cumulative data; Interactions between treatment and period (P<0.05). Mortality was analyzed as nonparametric data using NPar1Way of SAS.

Birds fed the restricted diet had decreased live gain (P<0.05) by 5.0% from day 1-43 compared to birds fed the adequate diet. Only birds fed 7.35×10¹ CFU/g Bacillus coagulans were no different (P>0.1) from the adequate diet fed birds, recovering 100% of lost live gain; there was also an additional numerical increase in live gain of 4.3% over the adequate diet. There were no other differences between Bacillus coagulans and restricted diet fed birds. Overall, there was no difference in feed efficiency; however, birds fed restricted diets had a numerically reduced gain/feed (5.1%; P>0.1). Birds fed 3.68×10¹ or 7.35×10¹ CFU/g Bacillus coagulans recovered 71.0% of lost feed efficiency. Supplementation of Bacillus coagulans at 7.35×10¹ improves growth and feed efficiency of birds fed nutritionally restricted diets.

TABLE 1
Power desired: 6.2, 6 treatments, 9 replicates, 43 d duration.

| Criteria | Grand Mean | Standard Deviation | % difference |
|---|---|---|---|
| Start weight | 41 | 0.1 | 0.29 |
| End weight | 2,544 | 164 | 7.56 |
| Cumulative | 2,435 | 176 | 8.50 |
| Live Gain | | | |
| Cumulative | 588 | 49 | 9.75 |
| Gain:Feed | | | |
| Cumulative | 4,154 | 335 | 9.47 |
| Feed Intake | | | |

TABLE 2a
Formulation of basal, negative control diet.

| | | Phase | | | |
|---|---|---|---|---|---|
| | | Starter, d 1-21 | | Finisher, d 22-42 | |
| | | Treatment | | | |
| | | A | B | A | B |
| Code | Ingredient | Adequate | Restricted | Adequate | Restricted |
| | Corn | 1,019.44 | 1,140.82 | 1,110.36 | 1,228.03 |
| | SBM | 682.50 | 621.05 | 600.64 | 542.57 |
| | DDGS | 100.00 | 100.00 | 100.00 | 100.00 |
| | Poultry M&BM | 50.00 | 50.00 | 50.00 | 50.00 |
| 404 | Soybean Oil | 70.00 | 10.17 | 70.00 | 10.12 |
| 650 | Limestone | 21.97 | 22.36 | 19.43 | 19.80 |
| 544 | MonoCal 21% P | 17.25 | 17.54 | 13.84 | 14.11 |
| 1218 | ADM Poultry VTM 88 | 12.50 | 12.50 | 12.50 | 12.50 |
| 780 | Salt | 7.49 | 7.52 | 7.04 | 7.06 |
| 1200 | DL Methionine | 7.27 | 6.71 | 6.60 | 6.01 |
| 1180 | L-Lysine | 4.92 | 4.88 | 4.07 | 4.45 |
| 1181 | Threonine | 2.21 | 1.98 | 1.56 | 1.49 |
| 1178 | L-Valine | 1.63 | 1.51 | 1.15 | 0.97 |
| 540 | Sodium Bicarb | 1.50 | 1.50 | 1.50 | 1.50 |
| 5464 | Manganese Sulfate | 0.61 | 0.61 | 0.62 | 0.63 |
| 554 | OptiPhos | 0.25 | 0.25 | 0.25 | 0.25 |
| 1690 | Zinc Oxide | 0.23 | 0.23 | 0.23 | 0.23 |
| 1194 | L-Isoleucine | 0.20 | 0.33 | 0.17 | 0.25 |
| 1011 | Copper Chloride 54% | 0.03 | 0.03 | 0.04 | 0.04 |
| | Total | 2,000 | 2,000 | 2,000 | 2,000 |
| | ME, Kcal/lb (Swine) | 1,505 | 1,430 | 1,508 | 1,433 |
| | Crude Protein, % | 22.25 | 21.14 | 20.50 | 19.48 |
| | AA, SID, % | | | | |
| | Lysine | 1.22 | 1.15 | 1.09 | 1.04 |
| | SAA | 0.91 | 0.86 | 0.84 | 0.79 |
| | Methionine | 0.63 | 0.59 | 0.58 | 0.54 |
| | Threonine | 0.82 | 0.77 | 0.73 | 0.69 |
| | Tryptophan | 0.26 | 0.25 | 0.24 | 0.23 |
| | Valine | 0.92 | 0.87 | 0.83 | 0.78 |
| | Isoleucine | 0.82 | 0.78 | 0.75 | 0.71 |
| | Leucine | 1.57 | 1.51 | 1.47 | 1.41 |
| | Arginine | 1.32 | 1.24 | 1.20 | 1.13 |

TABLE 2b
Experimental ingredients to be added at the expense of corn.

| Treatment | Inclusion/Product | Premix TBD |
|---|---|---|
| C. 3.68 × 10¹ | 0.02 lbs/ton | |
| CFU/g B. coagulans | | |
| D. 7.35 × 10¹ | 0.04 lbs/ton | |
| CFU/g B. coagulans | | |
| E. 1.10 × 10² | 0.06 lbs/ton | |
| CFU/g B. coagulans | | |
| F. 1.47 × 10² | 0.08 lbs/ton | |
| CFU/g B. coagulans | | |

TABLE 3
Effects of Bacillus coagulans fed to broilers on a nutritionally restricted diet^{1, 2, 3}.

| | | | | |
|---|---|---|---|---|
| | Live | Gain/ | Feed | |
| | Gain⁴ | Feed⁵ | Intake⁶ | Mortality⁷ |
| Treatment | (g) | (g) | (g) | (n) |

d 1-22

| | | | | |
|---|---|---|---|---|
| A. Adequate Diet | 984^{a} | 834^{a} | 1,177 | 8 |
| B. Restricted Diet | 884^{cd} | 747^{c} | 1,184 | 4 |
| C. B + 38.6 CFU/g | 909^{cd} | 774^{bc} | 1,175 | 6 |
| D. B + 73.5 CFU/g | 956^{ab} | 790^{b} | 1,211 | 4 |
| E. B + 110 CFU/g | 870^{d} | 750^{c} | 1,163 | 4 |
| F. B + 147 CFU/g | 920^{bc} | 780^{bc} | 1,184 | 3 |
| SEM | 17 | 12 | 23 | — |
| Treatment (P-value) | <0.001 | <0.001 | 0.713 | 0.695 |

d 23-43

| | | | | |
|---|---|---|---|---|
| A. Adequate Diet | 1,503 | 512 | 2,964 | 1 |
| B. Restricted Diet | 1,481 | 501 | 2,991 | 1 |
| C. B + 38.6 CFU/g | 1,542 | 528 | 2,925 | 0 |
| D. B + 73.5 CFU/g | 1,643 | 528 | 3,128 | 1 |
| E. B + 110 CFU/g | 1,515 | 527 | 2,873 | 0 |
| F. B + 147 CFU/g | 1,471 | 500 | 2,952 | 0 |
| SEM | 50 | 20 | 90 | — |
| Treatment (P-value) | 0.197 | 0.812 | 0.512 | 0.700 |

d 1-43

| | | | | |
|---|---|---|---|---|
| A. Adequate Diet | 2,490^{ab} | 610 | 4,088 | 9 |
| B. Restricted Diet | 2,365^{b} | 579 | 4,104 | 5 |
| C. B + 38.6 CFU/g | 2,451^{b} | 601 | 4,081 | 6 |
| D. B + 73.5 CFU/g | 2,598^{a} | 601 | 4,326 | 5 |
| E. B + 110 CFU/g | 2,378^{b} | 592 | 4,020 | 4 |
| F. B + 147 CFU/g | 2,381^{b} | 573 | 4,166 | 3 |
| SEM | 56 | 15 | 89 | — |
| Treatment (P-value) | 0.022 | 0.431 | 0.258 | 0.560 |

^{abcd}Within a column, in a timeframe, means without common superscripts differ, P < 0.05.
¹Data are means of 9 reps with 8 birds/pen (roosters) fed experimental diets for 43 d beginning at hatch.
²Treatments included: Adequate nutrition [Trt. A, positive control]; restricted nutrition [Trt. B, negative control]; or restricted with B. coagulans [Trts. C-F].
³The level of significance (P-value) accorded to the main effect of dietary treatment.
⁴Live gain = (pen weight period end − pen weight period start)/# birds at period end.
⁵Gain/Feed = g live gain/kg feed intake.
⁶Feed Intake = total feed disappearance per pen/# birds at period end.
⁷Mortality = exact count of instances.

TABLE 4
Repeated effects of B. coagulans fed to broilers on a nutritionally restricted diet^{1,2,3}.

| | Diet | | | | | | | | | |
|---|---|---|---|---|---|---|---|---|---|---|
| | Adequate | Restricted | | | | | | | | |
| | B. coag., CFU/g | | | | | | | | | |
| | — | — | 38.6 | 73.5 | 110 | 147 | | Level of Significance | | |
| | Trt | | | | | | | | | Trt % |
| | A | B | C | D | E | F | SEM | Trt | Period | Period |
| Body Wt, g | | | | | | | 31 | <0.001 | <0.001 | 0.006 |
| D 1 | 41 | 41 | 41 | 41 | 41 | 41 | 0 | 0.126 | | |
| D 8 | 205 | 201 | 206 | 207 | 204 | 209 | 4 | 0.772 | | |
| D 15 | 547^{x} | 512^{yz} | 517^{yz} | 530^{xyz} | 505^{z} | 533^{xy} | 10 | 0.080 | | |
| D 22 | 1,034^{a} | 927^{b} | 954^{b} | 999^{a} | 914^{b} | 952^{b} | 18 | <0.001 | | |
| D 29 | 1,648^{a} | 1,538^{b} | 1,533^{b} | 1,600^{a} | 1,524^{b} | 1,516^{b} | 27 | 0.001 | | |
| D 36 | 2,254^{a} | 2,128^{c} | 2,121^{c} | 2.234^{ab} | 2,138^{bc} | 2,097^{c} | 42 | 0.013 | | |
| D 43 | 2,599 | 2,499 | 2,530 | 2,669 | 2,511 | 2,471 | 54 | 0.102 | | |
| Live Gain, g | | | | | | | | 0.099 | <0.001 | 0.001 |
| D 1-8 | 159 | 159 | 164 | 165 | 161 | 166 | 4 | 0.816 | | |
| D 9-15 | 342^{a} | 305^{b} | 308^{b} | 320^{ab} | 301^{b} | 324^{ab} | 8 | 0.016 | | |
| D 16-22 | 474^{a} | 415^{b} | 421^{b} | 462^{a} | 408^{b} | 426^{b} | 13 | 0.001 | | |
| D 23-29 | 567^{x} | 556^{xy} | 545^{xyz} | 567^{x} | 527^{yz} | 516^{z} | 17 | 0.076 | | |
| D 30-36 | 607 | 590 | 588 | 635 | 614 | 581 | 25 | 0.676 | | |
| D 37-43 | 258^{z} | 300^{yz} | 409^{xy} | 453^{x} | 364^{xyz} | 374^{xy} | 50 | 0.093 | | |
| Gain/Feed, g/kg | | | | | | | | 0.328 | <0.001 | <0.001 |
| D 1-8 | 884^{b} | 927^{ab} | 990^{a} | 972^{a} | 937^{ab} | 989^{a} | 23 | 0.013 | | |
| D 9-15 | 876^{a} | 802^{c} | 807^{bc} | 824^{bc} | 807^{bc} | 831^{b} | 10 | <0.001 | | |
| D 16-22 | 761^{a} | 663^{b} | 692^{b} | 719^{ab} | 664^{b} | 677^{b} | 21 | 0.015 | | |
| D 23-29 | 668 | 635 | 640 | 644 | 632 | 599 | 17 | 0.116 | | |
| D 30-36 | 538 | 542 | 529 | 542 | 547 | 511 | 25 | 0.924 | | |
| D 37-43 | 300 | 311 | 426 | 435 | 394 | 394 | 50 | 0.239 | | |
| Feed Intake, g | | | | | | | | 0.287 | <0.001 | 0.618 |
| D 1-8 | 180 | 173 | 165 | 170 | 172 | 168 | 4 | 0.148 | | |
| D 9-15 | 390 | 380 | 381 | 388 | 373 | 389 | 8 | 0.592 | | |
| D 16-22 | 625 | 627 | 610 | 642 | 619 | 638 | 20 | 0.843 | | |
| D 23-29 | 849 | 878 | 854 | 882 | 828 | 865 | 22 | 0.572 | | |
| D 30-36 | 1,136 | 1,096 | 1,113 | 1,198 | 1,125 | 1,145 | 49 | 0.731 | | |
| D 37-43 | 901 | 951 | 959 | 1,046 | 925 | 942 | 38 | 0.170 | | |

### Example 4

### Evaluation of Efficacy of Bacillus coagulans DFM on the Growth Performance of Broilers

Experimental treatment groups included Treatment A: Positive control nutritionally adequate diet devoid of DFM; Treatment B: As A+7.35×10⁴ CFU/g Bacillus subtilis (1 lb/ton Novela; 73,500 CFU/g); Treatment C: Negative control nutritionally restricted diet devoid of DFM (5% reduction in CP+EAA); Treatment D: As C+7.35×10¹ CFU/g Bacillus coagulans (4 lb/ton MDG mix 2; 73.5 CFU/g); Treatment E: As C+7.35×10² CFU/g Bacillus coagulans (4 lb/ton MDG mix 3; 735 CFU/g); and Treatment F: As C+7.35×10³ CFU/g Bacillus coagulans (4 lb/ton MDG mix 4; 7,350 CFU/g).

Birds were assigned to pen based upon chick weight. Pens were then randomly allotted to dietary treatment and immediately started on the study. Pens remained on dietary treatment until the end of the experiment. Daily management followed standard operating procedures when feeding, watering, and monitoring for health decline in the birds. Unit lights were left on for the entire duration of the experiment. Unit temperature was set at 95° F. and lowered 1° daily starting from d 0 when they arrived in the barn. The room was maintained at 85° once the temperature was lowered to that point. Farm personnel inspected water troughs daily, and they cleaned and refreshed if needed by emptying the trough and refilling with fresh water. Pens were lined with 60 lb indented craft paper for the entire experiment to induce a health challenge. Litter from the previous experiment (16-P082, control diet) was used in all battery cages in an attempt to induce a health response. Approximately 35 g of used litter was placed in each cage, on top of the indented craft paper. Paper and litter were changed on d 14 at time of weight & feed recording.

Measurements included total pen weights (day 0, day 14, and day 20); feed disappearance (day 14 and day 20); feed/gain ratio (lb/lb and kcal/lb; day 1-14, day 14-20, and day 1-20; feed/gain can be adjusted for mortality with the following equation: total feed consumed/(pen live weight gain+mortality weight gain)); morbidity and mortality; and high and low temperatures (along with HOBO device)

Experimental test materials for diet formulation included Novela (MDG) and Bacillus coagulans (MDG). Diets remained the same throughout the trial. Final diets were manufactured from a basal diet with the addition of an experimental mixture at DEO. Four different concentrations of the product were manufactured for inclusion into the final diets. Premixes weighed 0.25 lbs., which could be included in their entirety into a 125 lb. treatment batch. The MDG product was premixed with an additional 5 lbs. of corn to ensure thorough mixing prior to addition into the final manufacture of the experimental diets.

Litter from the previous experiment was used in the battery cages in an attempt to induce an immune response. Approximately 30 g of used litter was placed in the cage, on top of the indented craft paper. Procedure for the collection of excreta and measurement of NH₃ (Trt. A & B) according to Hossain et al (2015) & Zhang et al (2013) as a pilot study for additional Novela work. Paper from excreta pan was removed and replaced w/clean indented craft paper on day 12. On day 15, 3 samples of 300 g of excreta were collected from 12 replicates for Treatments A & B and placed into plastic GLAD containers, where 2, 4, & 6 ounces of water were added. The samples were then sealed with Parafilm and allowed to ferment for 24 hrs. After 24 hours (day 16), samples were manually shaken for 30 seconds to break any possible crust formation, as well as homogenize the sample, prior to inserting the gas pump tube. The Gastec gas pump tube was then inserted into the containers through a hole previously sealed with adhesive tape. The gas pump tube was inserted into the container until the tip of the tube was approximately 2 cm above the material where the sample was procured. Personnel followed the aforementioned procedure for the NH₃ reading at 96 hrs or day 19 of the trial.

Initial data analysis was performed for all metrics to determine normality of distribution and outliers (±>3 standard deviations from the grand mean). Statistical (SAS) procedures utilized “Pen” as the experimental unit, “Treatment” as the fixed effect, and “Replicate” as the random effect. Periodic & cumulative gain, feed intake, and feed efficiency: PROC MIXED. Periodic & cumulative mortality: PROC NPAR1WAY.

Birds on trial were fed either a control diet with or without Novela, or a nutrient restricted diet with or without 1 of 3 concentrations of Bacillus coagulans. Birds were healthy with relatively low mortality (only 6.6%). There were no issues with diets or bird handling throughout the trial.

From day 1-14 there was no difference between birds fed the control diet with or without Novela. Birds fed a restricted diet had decreased (P<0.01) BW and live gain by 13.9 and 15.1%, respectively, and reduced (P<0.01) feed efficiency and MAFCR by 7.3 and 7.5%, respectively. Inclusion of 73.50, 735.0, and 7,350 CFU/g Bacillus coagulans allowed for recovery of 60.2, 20.0, and 36.7%, respectively, of lost BW, and recovered of 59.6, 19.3, and 36.8%, respectively, of live gain lost. Inclusion of 73.50, 735.0, and 7,350 CFU/g Bacillus coagulans allowed for recovery of 47.5, 34.4, and 54.1%, respectively, of lost feed efficiency, and recovered 55.6, 33.3, and 55.6%, respectively, of lost MAFCR.

From day 15-20 there was no difference between birds fed the control diet with or without Novela. Birds fed a restricted diet had decreased (P<0.01) BW, live gain, total gain, and feed intake by 11.5, 7.8, 6.6, and 6.1%, respectively. Inclusion of 73.50 CFU/g Bacillus coagulans allowed for recovery of 82.1, 140.9, 133.3, and 137.5% lost BW, live gain, total gain, and feed intake, respectively. Inclusion of 735.0 CFU/g Bacillus coagulans reduced (P<0.05) live gain, total gain, and feed intake 10.8, 11.8, and 6.2%, respectively, compared to restricted nutrient fed birds. There was no difference between restricted fed birds and birds receiving 7,350 CFU/g Bacillus coagulans.

From day 1-20, there was no difference between birds fed the control diet with our without Novela. Birds fed a restricted diet had decreased (P<0.01) live gain by 12.0% (79 g) compared to birds fed the control diet. Inclusion of 73.50 CFU/g Bacillus coagulans recovered (P<0.05) 82.3% of live gain lost in restricted fed birds. Inclusion of 7,350 CFU/g Bacillus coagulans numerically recovered (P>0.05) 22.8% of live gain lost in restricted fed birds. Inclusion of 735.0 CFU/g Bacillus coagulans numerically reduced (P>0.05) live gain by 4.5% compared to restricted fed birds.

There was no difference with the inclusion of Novela into control diets; however, feed efficiency was numerically increased 3% compared to the control diets, similar to previous research. Inclusion of just 73.50 CFU/g Bacillus coagulans into a nutrient restricted diet allowed for recovery of lost BW, gain, and feed efficiency to levels not different from the control diet. Birds fed 73.5 CFU/g B. coagulans had increased gain compared to birds fed a nutritionally restricted diet devoid of DFMs. Bacillus coagulans microbes added to the broiler diet appear to produce enzymes to increase protein and fat digestibility.

TABLE 5
Formulation of basal, negative control diet.

| | Trt A | Trt C |
|---|---|---|
| Ingredient | Positive Control | Negative Control |
| Corn | 1,095.84 | To 2,000 |
| SBM | 664.06 | 611.22 |
| DDGS | 100.00 | 100.00 |
| Poultry Meat & Bone Meal | 50.00 | 50.00 |
| Ground Limestone | 24.39 | 24.68 |
| Mono-Cal 21% | 19.23 | 19.57 |
| ADM Poultry VTM 88 | 12.50 | 12.50 |
| Soybean Oil | 10.00 | 10.00 |
| Salt | 7.49 | 7.52 |
| DL-Methionine | 6.92 | 6.40 |
| L-Lysine | 4.86 | 4.93 |
| Threonine | 1.56 | 1.52 |
| Sodium Bicarb | 1.50 | 1.50 |
| L-Valine | 1.40 | 1.27 |
| OptiPhos | 0.25 | 0.25 |
| Novela | +/− | |
| MDG mix (as specified in Table 2) | | +/− |
| Total | 2,000 | 2,000 |

Nutrient Specifications

| | | |
|---|---|---|
| Crude Protein, % | 22.00 | 20.90 |
| Crude Fat, % | 3.47 | 3.48 |
| ADF, % | 4.68 | 4.56 |
| Lysine, Total, % | 1.39 | 1.32 |

Essential AAs, Dig, %

| | | |
|---|---|---|
| Lysine | 1.20 | 1.14 |
| SAA | 0.89 | 0.84 |
| Threonine | 0.78 | 0.74 |
| Tryptophan | 0.26 | 0.25 |
| Valine | 0.90 | 0.85 |
| Isoleucine | 0.80 | 0.76 |
| Arginine | 1.30 | 1.22 |
| Phosphorus, Available, % | 0.48 | 0.48 |
| Calcium, Total, % | 0.98 | 0.98 |
| Sodium, % | 0.20 | 0.20 |

TABLE 6
Experimental ingredients to be added at the expense of corn.

| Treatment | Inclusion/Product | Add to 125# Diet |
|---|---|---|
| B. 7.35 × 10⁴ CFU/g B. subtilis | 1 lb/ton Novela | 0.07 lbs. |
| D. 73.50 CFU/g B. coagulans | 4 lb/ton MDG mix 2 | 0.25 lbs. |
| E. 735.0 CFU/g B. coagulans | 4 lb/ton MDG mix 3 | 0.25 lbs. |
| F. 7350 CFU/g B. coagulans | 4 lb/ton MDG mix 4 | 0.25 lbs. |

TABLE 7
Effects of DFM supplementation on growth performance of broilers¹.

| | | | | |
|---|---|---|---|---|
| | Live | Total | Feed | Gain/ |
| | Gain² | Gain³ | Intake⁴ | Feed⁵ |
| Treatment | (g) | (g) | (g) | (g/kg) |

d 1-14

| | | | | |
|---|---|---|---|---|
| A. Control Diet | 377^{a} | 331 | 395 | 836^{a} |
| B. A + 1 lb/ton Novela | 365^{ab} | 354 | 429 | 827^{ab} |
| C. Restricted Diet | 320^{e} | 314 | 405 | 775^{a} |
| D. C + 73.5 CFU/g B. coag | 354^{bc} | 338 | 420 | 804^{c} |
| E. C + 735 CFU/g B. coag | 331^{de} | 326 | 410 | 796^{c} |
| F. C + 7350 CFU/g B. coag | 341^{cd} | 341 | 422 | 808^{bc} |
| SEM | 7 | 12 | 13 | 8 |

P-values

| | | | | |
|---|---|---|---|---|
| Treatment | <0.001 | 0.281 | 0.508 | <0.001 |

d 15-21

| | | | | |
|---|---|---|---|---|
| A. Control Diet | 281^{ab} | 273^{ab} | 395^{ab} | 694 |
| B. A + 1 lb/ton Novela | 282^{ab} | 282^{a} | 389^{ab} | 724 |
| C. Restricted Diet | 259^{bc} | 255^{b} | 371^{abc} | 696 |
| D. C + 73.5 CFU/g B. coag | 290^{a} | 279^{ab} | 404^{a} | 691 |
| E. C + 735 CFU/g B. coag | 231^{d} | 225^{c} | 348^{c} | 647 |
| F. C + 7350 CFU/g B. coag | 256^{c} | 256^{b} | 369^{bc} | 691 |
| SEM | 8 | 9 | 12 | 21 |

P-values

| | | | | |
|---|---|---|---|---|
| Treatment | <0.001 | 0.001 | 0.023 | 0.318 |

d 1-20

| | | | | |
|---|---|---|---|---|
| A. Control Diet | 658^{a} | 571 | 790 | 719 |
| B. A + 1 lb/ton Novela | 623^{ab} | 605 | 817 | 738 |
| C. Restricted Diet | 579^{cd} | 564 | 776 | 728 |
| D. C + 73.5 CFU/g B. coag | 644^{a} | 581 | 812 | 712 |
| E. C + 735 CFU/g B. coag | 553^{d} | 533 | 752 | 707 |
| F. C + 7350 CFU/g B. coag | 597^{bc} | 597 | 791 | 754 |
| SEM | 15 | 27 | 21 | 22 |

P-values

| | | | | |
|---|---|---|---|---|
| Treatment | <0.001 | 0.489 | 0.273 | 0.687 |

¹Data are means of 8 replicates of 6 birds/pen fed experimental diets starting on d 0 to d 20. Experimental period began immediately after arrival to the DEO Poultry Unit. Data were analyzed as a randomized complete design using the Mixed Procedure of SAS.
²Live gain was calculated as pen weight at the end of the period minus pen weight at the start of the period, divided by the number of birds in the pen at the end of the experimental period.
³Total gain was calculated as pen weight at the end of the period, plus dead bird weight, minus pen weight at the start of the period, divided by the number of birds in the pen at the beginning of the experimental period.
⁴Feed Intake is representative of the total feed disappearance per pen, divided by the number of birds in the pen at the end of the experimental period.
⁵Gain/Feed was calculated as the dividend of gain by kg feed intake.

TABLE 8
Effects of DFM supplementation on body
weight and mortality of broilers¹.

| | | | | |
|---|---|---|---|---|
| | Start | End | Mortal- | |
| | Wt | Wt² | ity | MAFCR³ |
| Treatment | (g) | (g) | (n) | (g/g) |

d 1-14

| | | | | |
|---|---|---|---|---|
| A. Control Diet | 29.0 | 405.8^{a} | 6 | 1.20^{d} |
| B. A + 1 lb/ton Novela | 29.0 | 393.6^{ab} | 2 | 1.21^{cd} |
| C. Restricted Diet | 28.9 | 349.2^{e} | 1 | 1.29^{a} |
| D. C + 73.5 CFU/g B. coag | 28.9 | 383.3^{bc} | 4 | 1.24^{b} |
| E. C + 735 CFU/g B. coag | 28.9 | 360.5^{de} | 1 | 1.26^{b} |
| F. C + 7350 CFU/g B. coag | 28.9 | 370.0^{cd} | 0 | 1.24^{bc} |
| SEM | 0.04 | 7.2 | — | 0.01 |

P-values

| | | | | |
|---|---|---|---|---|
| Treatment | 0.326 | <0.001 | 0.047 | <0.001 |

d 15-20

| | | | |
|---|---|---|---|
| A. Control Diet | 686.8^{a} | 1 | 1.46 |
| B. A + 1 lb/ton Novela | 672.7^{a} | 0 | 1.38 |
| C. Restricted Diet | 608.1^{b} | 1 | 1.45 |
| D. C + 73.5 CFU/g B. coag | 672.7^{a} | 1 | 1.46 |
| E. C + 735 CFU/g B. coag | 593.9^{b} | 2 | 1.55 |
| F. C + 7350 CFU/g B. coag | 625.7^{b} | 0 | 1.45 |
| SEM | 12.3 | | 0.05 |

P-values

| | | | |
|---|---|---|---|
| Treatment | <0.001 | 0.611 | 0.337 |

d 1-20

| | | | | |
|---|---|---|---|---|
| A. Control Diet | 29.0 | 686.8^{a} | 7 | 1.41 |
| B. A + 1 lb/ton Novela | 29.0 | 672.7^{a} | 2 | 1.38 |
| C. Restricted Diet | 28.9 | 608.1^{b} | 2 | 1.38 |
| D. C + 73.5 CFU/g B. coag | 28.9 | 672.7^{a} | 5 | 1.42 |
| E. C + 735 CFU/g B. coag | 28.9 | 593.9^{b} | 3 | 1.42 |
| F. C + 7350 CFU/g B. coag | 28.9 | 625.7^{b} | 0 | 1.33 |
| SEM | 0.04 | 12.3 | — | 0.05 |

P-values

| | | | | |
|---|---|---|---|---|
| Treatment | 0.326 | <0.001 | 0.126 | 0.767 |

¹Data are means of 8 replicates of 6 birds/pen fed experimental diets starting on d 0 to d 20. Experimental period began immediately after arrival to the DEO Poultry Unit. Data were analyzed as a randomized complete design using the Mixed Procedure of SAS.
²End wt was calculated as the dividend of total pen weight and number of birds in the pen at the end of the period.
³Mortality adjusted feed conversion rate (FCR) was calculated as the dividend of feed intake and total gain during the experimental period.

TABLE 9
The effects of Novela on ammonia volatilization.

| | |
|---|---|
| Ctrl (ppm) | Novela (ppm) |

| Rep | 3M | 3La | 3M | 3La | H2O (oz) |
|---|---|---|---|---|---|
| 1 | 49 | 36 | 25 | 34 | 2 |
| 1 | 40 | 34 | 20 | 23 | 4 |
| 1 | 30 | 27 | 20 | 18 | 6 |
| 2 | 30 | — | 35 | — | 2 |
| 2 | 30 | — | 25 | — | 4 |
| 2 | 25 | — | 15 | — | 6 |

*Samples were collected on May 24, 2016 w/a room temperature of 85.5° F, and barometric pressure on 1,021 hpa.

1. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof, wherein the Bacillus strain causes an effect selected from the group consisting of improving the performance of the animal and improving the health of the animal, and combinations thereof.

2. The method of claim 1 wherein the improvement to the health of the animal is reduced bioburden in the animal.

3. The method of claim 1 wherein the improvement to the health of the animal is increasing the immune response in the animal.

4. The method of claim 1 wherein the strain administered is Bacillus strain BC1 (NRRL No. B-67744).

5. The method of claim 1 wherein the strain administered is Bacillus strain BCP2 (NRRL No. B-67745).

6. The method of claim 1 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.

7. A commercial package, feed additive for an animal feed, additive for the drinking water of an animal or animal feed composition, said commercial package, feed additive, feed composition, additive for the drinking water of the animal or animal feed composition comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

8. The commercial package, feed additive, feed composition, additive for the drinking water of the animal or animal feed composition of claim 7 wherein the Bacillus strain causes an effect selected from the group consisting of improving the performance of the animal, improving the health of the animal, and combinations thereof.

9. The commercial package, feed additive, additive for the drinking water of the animal, or feed composition of claim 7 further comprising a carrier for the Bacillus strains.

10. The commercial package, feed additive, additive for the drinking water of the animal, or feed composition of claim 9 wherein the carrier is selected from the group consisting of a bran, rice hulls, a salt, mineral oil, a dextrin, whey, sugar, limestone, dried starch, sodium silico aluminate, vegetable oil, and combinations thereof.

11. The commercial package, feed additive, additive for the drinking water of the animal, or feed composition of claim 7 further comprising a binder.

12. The commercial package, feed additive, additive for the drinking water of the animal, or feed composition of claim 11 wherein the binder is selected from the group consisting of clay, yeast cell wall components, aluminum silicate, glucan, and combinations thereof.

13. The commercial package, feed additive, additive for the drinking water of the animal, or feed composition of claim 7 in the form of a dietary nutrient composition.

14. A method of feeding an animal, the method comprising the step of administering to the animal a feed composition or drinking water comprising an effective amount of an additive comprising an isolated Bacillus coagulans strain selected from the group consisting of Bacillus strain BC1 (NRRL No. B-67744), Bacillus strain BCP2 (NRRL No. B-67745), a strain having all of the identifying characteristics of Bacillus strain BC1 (NRRL No. B-67744), a strain having all of the identifying characteristics of Bacillus strain BCP2 (NRRL No. B-67745), and combinations thereof.

15. The method of claim 14 wherein the strain administered is Bacillus strain BC1 (NRRL No. B-67744).

16. The method of claim 14 wherein the strain administered is Bacillus strain BCP2 (NRRL No. B-67745).

17. The method of claim 14 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™, and vancomycin.

18. The method of claim 14 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
